(19) United States
(12) Patent Application Publication
(10) Pub. No.: US 2021/0246456 A1
(43) Pub. Date: Aug. 12, 2021

(54) USE OF THE U94 MOLECULE OF HUMAN HERPESVIRUS 6 AND DERIVATIVES THEREOF TO INCREASE OR INDUCE THE EXPRESSION OF THE HLA-G MOLECULE

(71) Applicant: FONDAZIONE U. BONINO E M.S. PULEJO, Messina (IT)

(72) Inventors: Arnaldo CARUSO, Brescia (IT); Roberta RIZZO, Ferrara (IT); Dario DI LUCA, Masi Torello (Ferrara) (IT); Elisabetta CASELLI, Ferrara (IT); Francesca CACCURI, Trebisacce (Cosenza) (IT)

(21) Appl. No.: 16/972,897

(22) PCT Filed: Jun. 7, 2019
(86) PCT No.: PCT/IB2019/054749

§ 371 Date: Dec. 7, 2020

(30) Foreign Application Priority Data
Jun. 8, 2018 (IT) 102018000006137

(51) Int. Cl. C12N 15/67; C12N 15/86

(52) U.S. Cl. CPC C12N 15/67; C12N 2710/16541; C12N 2710/16522; C12N 15/86

(57) ABSTRACT

A method of enhancing or inducing the expression of the HLA-G molecule by human cells is provided. The method includes administering the human Herpesvirus 6 U94 molecule or a derivative thereof, or a gene expression vector including and expressing the human Herpesvirus 6 U94 gene to a patient in need thereof or to an in vitro cell, tissue, or organ culture.

The present invention relates to a new medical use of the human Herpesvirus 6 U94 molecule and derivatives thereof.

### BACKGROUND OF THE INVENTION

### HLA-G Antigen

HLA (Human Leukocyte Antigen)-G is a non-classical HLA class I antigen, featuring a low allelic polymorphism, compared to the other classical class I HLAs, and a restricted tissue distribution. 7 Isoforms have been identified (membrane-bound isoforms: HLA-G1, G2, G3, G4, soluble isoforms: HLA-G5, G6, G7), obtained by alternative mRNA splicing.

The inhibitory action of HLA-G molecules occurs through interaction with inhibitory receptors LILRB1 (leukocyte immunoglobulin-like receptor, subfamily B, member 1 (LILRB1)/immunoglobulin-like transcript 2 (ILT2)/CD85j ILT-2), LILRB2 (subfamily B, member 2 (LILRB2)/immunoglobulin-like transcript 4 (ILT4)/CD85d) and killer cell immunoglobulin-like receptor KIR2DL4. KIR2DL4 is predominantly expressed by decidual NK cells, LILRB1 is expressed on B cells, some T cells and NK cells, all monocytes and dendritic cells, while LILRB2 is myeloid-specific and only expressed by monocytes and dendritic cells (Rizzo R, Bortolotti D, et al. New insights into HLA-G and inflammatory diseases. Inflamm Allergy Drug Targets. 2012; 11(6):448-63).

The HLA-G antigen was initially identified as a molecule selectively expressed at the maternal-fetal interface, on the surface of cytotrophoblasts, where, together with the presence of the HLA-C and HLA-E molecules, it plays an immunoregulatory role, by inhibiting the lytic action of NK cells and affecting cytokine expression (Lynge Nilsson L, Djurisic S, Hviid T V. Controlling the Immunological Crosstalk during Conception and Pregnancy: HLA-G in Reproduction. Front Immunol. 2014; 5:198).

Subsequently, the presence of HLA-G molecules was detected under physiological conditions in a subpopulation of peripheral blood CD14-positive monocytes and in thymic epithelial cells. The expression thereof is enhanced by Interleukin-10, interferons, and hormones. HLA-G expression has two effects: i) in the short term, it inhibits immune effector cells (natural killer (NK) cells; cytotoxic T lymphocytes, dendritic cells (DC), and ii) in the long term, it induces immunoregulatory cells (HLA-G+ regulatory T (Treg) cells), CD4low or CD8low T suppressor cells, Treg type 1 (Tr1) and DC-10 (Carosella E D, Gregori S et al. The tolerogenic interplay(s) among HLA-G, myeloid APCs, and regulatory cells. Blood 2011; 118: 6499-505). For this reason, selective induction of HLA-G molecules can create a tolerogenic environment that can be used in physiological and pathological contexts.

The HLA-G molecule is a powerful immunomodulator, with inhibitory functions against the immune system. In contrast, no stimulatory functions or allogeneic responses are known.

Tissues constitutively expressing HLA-G are considered as immune-privileged tissues, that is particular districts of the body such as the brain, the maternal-fetal interface, the testicles, the eye, the thymus, in which an inflammatory response would lead to irreparable damage.

HLA-G expression was subsequently detected in pathological contexts such as neoplasms, viral infections and inflammatory disease (Alegre E, Rizzo R, et al. Some basic aspects of HLA-G biology. J Immunol Res. 2014; 2014: 657625). In these conditions, HLA-G production was observed in different cell types: tumour cells, monocytes/macrophages, dendritic cells, CD8+ and CD4+ T cells, myocardial and striated muscle cells, neuronal cells, epithelial and thymus cells.

### Immunosuppressive Therapy in Transplants and Autoimmune Diseases

Transplants of solid organs and hematopoietic stem cells (HSCT) are both life-saving therapies for patients with irreversible damage to organs or severe hematopoietic diseases. Incompatibility between donor and recipient, in particular between HLA class I and II genes, leads to alloresponse of the innate and adaptive immune system, which must be controlled with immunosuppressive drugs. Immunosuppressive therapy is initiated during surgery and must be maintained throughout life, following established therapeutic schedules. Autoimmune diseases occur when the immune system identifies the body's own cells as foreign. The resulting immune response can cause irreparable damage to organs and tissues. For example, in multiple sclerosis, the myelin coating insulating the nerve cells is damaged. Currently, therapies to treat patients with autoimmune disease involve the use of immune suppressors, which help reduce the inflammatory attack on tissues.

Immune tolerance therapies are designed to stop or even prevent autoimmune disease while leaving intact the ability to fight body diseases. These tolerance therapies substantially reprogram the immune system, so that a short course of treatment will have lasting effects. Although immune tolerance therapies are mainly experimental, the Immune Tolerance Network (ITN) believes that targeted reprogramming of the immune system is extremely promising to effectively treat autoimmune diseases with fewer side effects than the current drugs.

In fact, current drugs exhibit numerous side effects, in particular the increase in infection incidence and tumour development. Thus, successful transplantation depends on the balance between rejection and side effects of the immunosuppressive therapies.

### Immune Inhibitory Therapy-Related Problems

Patients with solid organ (kidney, liver, heart, lung, pancreas, intestine) and bone marrow transplants must take immunosuppressant drugs daily to prevent acute and chronic rejection. Patients suffering from autoimmune diseases must take immune inhibiting drugs in order to control the disease.

The use of these drugs has been, and still is, one of the most complex aspects of post-transplant clinical management of subjects with autoimmune diseases. In fact, their incorrect use may on the one hand increase the incidence of acute and chronic rejections, on the other, the risk of morbidity and mortality from infections, neoplasms, cardiovascular disease and nephrotoxicity.

The narrow therapeutic range that distinguishes the use of these drugs (understood as the ease of passing from a toxicity to an underdosing condition), has triggered numerous pharmacokinetic, pharmacodynamic and clinical studies, with the aim of identifying the most effective and safest methods of use.

The results, in addition to confirming the narrow therapeutic range, have also shown highly variable pharmacokinetics, as they can be affected by several factors, such as age, sex, race, obesity, metabolic disease, renal disease, genetic factors, etc. Another aspect of great importance for its clinical implications concerns pharmacological interactions, documented both among some of the immunosuppressive drugs and between these and other widely used drugs, such as antifungals, some antibiotics, some antiepileptics. In these cases, to avoid the risk of over- or under-dosing, changing the daily dosage, even drastically, becomes essential.

The reasons for the complexity of the immunosuppressive therapy therefore depend on i) the narrow therapeutic range; ii) variable pharmacokinetics between individuals iii) the presence of significant drug interactions.

### HLA-G and Transplants

The development of a certain level of tolerance against allogeneic antigens can favour the prognosis of the transplant. In solid organs, induction of tolerance can decrease the risk of acute and chronic rejection of the transplant and improve survival. In HSCT, tolerance can reduce the graft-versus-host and host-versus-graft disease.

The search for natural tolerance mediators represents an approach for defining new strategies in order to offer effective therapies to transplanted patients.

Decades of research have identified HLA-G as a natural molecule capable of inducing tolerance.

The first evidence of the involvement of HLA-G expression in transplants has been reported in heart transplantation (Lila N, Carpentier A, et al. Implication of HLA-G molecule in heartgraft acceptance The Lancet 2000; 9221: 2138). In 36 heart transplant recipients, HLA-G expression in endomyocardial biopsies or serum was associated with a decrease in acute and chronic rejection episodes. These results were confirmed by other studies, which indicated that HLA-G expression improves the prognosis of heart transplant recipients (Lila N, Amrein C, et al. Human leukocyte antigen-G expression after heart transplantation is associated with a reduced incidence of rejection Circulation, 2002; 105: 1949-1954; Luque J, Torres M I, et al. Soluble HLA-G in heart transplantation: their relationship to rejection episodes and immunosuppressive therapy. Human Immunol, 2006; 67: 257-263; Sheshgiri R, Rao V, et al. Association between HLA-G expression and C4d staining in cardiac transplantation Transplantation, 2010; 89: 480-481).

In kidney, lung, liver-kidney, and kidney-pancreas transplants, increased HLA-G expression was associated with better engraftment (Le Rond S, Le Maoult J, et al. Alloreactive CD4+ and CD8+ T cells express the immunotolerant HLA-G molecule in mixed lymphocyte reactions: in vivo implications in transplanted patients. European J Immunol, 2004; 34: 649-660; Creput C, Durrbach A, et al. Human leukocyte antigen-G (HLA-G) expression in biliary epithelial cells is associated with allograft acceptance in liver-kidney transplantation. J Hepatol, 2003; 39: 587-594; Brugiere O, Thabut G, et al, Immunohistochemical study of HLA-G expression in lung transplant recipients The American J Transplant, 2009; 9: 1427-1438; Naji A, Le Rond S, et al. CD3+CD4 low and CD3+CD8 low are induced by HLA-G: novel human peripheral blood suppressor T-cell subsets involved in transplant acceptance. Blood 2007; 110: 3936-3948; Zarkhin V, Talisetti A, et al. Expression of soluble HLAG identifies favorable outcomes in liver transplant recipients,” Transplantation 201; 90: 1000-1005; Rebmann V, Bartsch D, et al. Soluble total human leukocyte antigen class I and human leukocyte antigen-G molecules in kidney and kidney/pancreas transplantation. Human Immunol, 2009; 70: 995-999).

Many studies analysed the role of HLA-G in HSCT. Increased HLA-G levels are positively correlated with a favourable transplant prognosis (LeMaux A, Noel G, et al. Soluble human leucocyte antigen-G molecules in peripheral blood haematopoietic stem cell transplantation: a specific role to prevent acute graft versus-host disease and a link with regulatory T cells,” Clinical Experimental Immunol, 2008; 152: 50-56; Waterhouse M, Duque-Afonso J, R. et al. Soluble HLA-G molecules and HLA-G 14-base pair polymorphism after allogeneic hematopoietic cell transplantation. Transplantation Proceed, 2013; 45: 397-401).

### HLA-G and Autoimmune Diseases

Several studies in recent years have shown that HLA-G plays a major role in controlling autoimmune/inflammatory diseases, such as multiple sclerosis (MS) (Rizzo R, Bortolotti D et al. HLA-G molecules in autoimmune diseases and infections. Frontiers in Immunology. 2014; 5,592), Crohn's disease (CD) (Rizzo R, Melchiorri L et al. Different production of soluble HLA-G antigens by peripheral blood mononuclear cells in ulcerative colitis and Crohn's disease: a noninvasive diagnostic tool? Inflammatory Bowel Diseases. 2008; 14(1):100-105), psoriasis (Aractingi S., Briand N et al. HLA-G and NK receptor are expressed in psoriatic skin: a possible pathway for regulating infiltrating T cells? American Journal of Pathology. 2001; 159(1):71-77), pemphigus (Gazit E., Slomov Y et al. HLA-G is associated with pemphigus vulgaris in Jewish patients. Human Immunology 2004; 65(1):39-46), celiac disease (Tones M., Lopez-Casado M et al. New advances in coeliac disease: serum and intestinal expression of HLA-G. International Immunology. 2006; 18(5):713-718), systemic lupus erythematosus (SLE) (Rizzo R., Hviid T et al. HLA-G genotype and HLA-G expression in systemic lupus erythematosus: HLA-G as a putative susceptibility gene in systemic lupus erythematosus. Tissue Antigens. 2008; 71(6):520-529), asthma (Rizzo R., Mapp C et al. Defective production of soluble HLA-G molecules by peripheral blood monocytes in patients with asthma. The Journal of Allergy and Clinical Immunology. 2005; 115(3):508-513), juvenile idiopathic arthritis (Prigione I, Penco F et al. HLA-G and HLA-E in patients with juvenile idiopathic arthritis. Rheumatology. 2011; 50(5):966-972), and rheumatoid arthritis (RA) (Rizzo R, Farina I et al. HLA-G may predict the disease course in patients with early rheumatoid arthritis. Human Immunology. 2013; 74(4):425-432).

Favoino et al. analysed sHLA-G in the serum of patients with systemic sclerosis (SSc). They divided the patients based on sHLA-G levels in the high and low production HLA-G groups and found greater disease severity correlated with low HLA-G levels (Favoino E, Favia I et al. Clinical correlates of human leucocyte antigen (HLA)-G in systemic sclerosis. Clinical and Experimental Immunology. 2015; 181(1):100-109). Zidi et al. observed higher sHLA-G levels in CD patients compared to controls. sHLA-G dimers were found in the advanced stage of the disease, thus suggesting a role for sHLA-G as a prognostic marker for progressive disease in patients with CD (Zidi I, Ben Yahia H et al. Association between sHLA-G and HLA-G 14-bp deletion/insertion polymorphism in Crohn's disease. International Immunology. 2015; 27(6):289-296). The sHLA-G dimers were also analysed by Fainardi et al. in multiple sclerosis, showing that HLA-G dimers in the cerebrospinal fluid were more frequent in patients with multiple sclerosis than in controls and in the MRI inactive disease, thus suggesting that HLA-G dimers may be involved in controlling the inflammatory response in multiple sclerosis (Fainardi E, Bortolotti D et al. Cerebrospinal fluid amounts of HLA-G in dimeric form are strongly associated to patients with MRI inactive multiple sclerosis. Multiple Sclerosis Journal. 2016; 22(2):245-249).

### HLA-G and Pregnancy

The important role of HLA-G in pregnancy has been fully characterized in recent years. Several HLA-G isoforms are expressed by trophoblastic cells at the maternal-fetal interface. HLA-G is expressed and released by trophoblastic cells and can interact with cell receptors expressed by immune cells (T cells, NK cells, macrophages, and dendritic cells) and non-immune (endothelial) cells present in the decidua, thereby activating inhibitory or activating signals. These interactions can (i) limit the maternal immune response against the semi-allogeneic fetal tissues by inhibiting the cytotoxicity of the decidual NK cells and T and B cell proliferation with induction of apoptosis of activated CD8+ T cells, (ii) stimulate placental development through secretion of pro-angiogenic factors, and (iii) provide a protective effect for the outcome of pregnancy by stimulating IL-4 secretion from CD4+ T cells. In pathological pregnancies (recurrent spontaneous abortions, preeclampsia, recurrent implantation failure), HLA-G expression levels are significantly reduced (Rizzo R, Vercammen M et al. The importance of HLA-G expression in embryos, trophoblast cells, and embryonic stem cells. Cell Mol Life Sci. 2011; 68(3):341-52), suggesting a pathogenetic involvement thereof.

### Clinical Use of HLA-G

Synthetic dimer peptides capable of binding the ILT4 receptor have been used in the treatment of skin transplanted mice, obtaining complete tolerance (LeMaoult J, Daouya M, et al. Synthetic HLA-G proteins for therapeutic use in transplantation. FASEB J, 2013; 27: 3643-3651). Mesenchymal stromal cell-derived exosomes containing high concentrations of HLA-G have been used for the treatment of a patient suffering from a grade IV graft-versus-host disease (Kordelas L, Rebmann V, et al. MSC-derived exosomes: a novel tool to treat therapy-refractory graft-versus-host disease. Leukemia, 2014; 28(4):970-3). After treatment, the patient showed an improvement in symptoms without adverse events.

### DESCRIPTION OF THE INVENTION

Despite the importance of the HLA-G molecule in regulating the immune response has been extensively studied and demonstrated, many problems remain unsolved for the use of this molecule in the therapeutic field.

The first problem is due to the trimolecular nature of HLA-G, formed by the heavy chain, beta 2 microglobulin and the peptide. In addition to being difficult to manufacture, this structure has low stability.

Moreover, the data available today is based on synthetic molecules or on molecules purified from cell line cultures transfected with the HLA-G gene. Interaction between HLA-G molecules (multimers) and association with other molecules are therefore not taken into consideration. For this reason, the development of a therapy-compatible HLA-G molecule is still progressing slowly.

The object of the present invention is to overcome these and other drawbacks of the prior art.

To this end, a first aspect of the present invention is the human Herpesvirus 6 U94 molecule or a derivative thereof, for use in a therapeutic immuno-suppression and/or anti-inflammatory treatment method, said method comprising administering to a subject an amount of U94 or derivatives thereof which is effective in modulating, i.e. enhancing or inducing HLA-G expression in said subject. The subject is preferably a mammal, more preferably a human being.

In the scope of the present description, the term “modulating” is sometimes used with reference to HLA-G expression in mammalian cells and/or tissues. It should be emphasized that, in this context, this term has the meaning of “enhancing or inducing”. The same applies to the variant “modulation”, which has the meaning of “enhancement or induction”.

The present invention is extremely advantageous compared to the state of the art, since an HLA-G molecule directly produced by the cells/tissues of the organism to be treated greatly facilitates the use of this protein for therapeutic purposes. In this way, synthesis and extraction problems are avoided and a molecule fully compatible with the parent cells/tissue is obtained. Moreover, thanks to the invention, the HLA-G molecules are induced both as membrane proteins and in the soluble form, with the advantages of both a local and a systemic action.

The HLA-G molecule produced by means of the present invention was shown to have a tolerogenic function, by inhibiting the cytotoxic activity of NK cells and cytotoxic T lymphocytes, the allogeneic T lymphocyte response, dendritic cell maturation, and by inducing the maturation of regulatory T cells and IL-10-expressing dendritic cells.

The modulation of the HLA-G molecule according to the invention can be used together with drugs capable of regulating the immuno-inhibitory response. This benefits the patient by reducing the required immunosuppressive drug levels and those required to induce better tolerance.

Accordingly, a second aspect of the invention is the human Herpesvirus 6 U94 molecule or a derivative thereof, for the above indicated use, as an adjuvant, in the therapeutic immunosuppressive treatment, co-administered with an immunosuppressive drug such as, but not limited to, FK506, ciclosporin, cortisone, mycophenolate, azathioprine, or everolimus.

Pharmaceutical preparations containing the active ingredients combined with suitable pharmaceutically acceptable excipients and/or carriers are conveniently used for administering the U94 molecule and derivatives thereof. The concentration of the active ingredients must be sufficient to obtain the modulation of the HLA-G molecule with an immuno-inhibiting effect. The selection of excipients and/or carriers to be used in the pharmaceutical preparations of the invention is within the skills of the person skilled in the art.

In the scope of the present invention, the human Herpesvirus 6 U94 molecule can be administered as a protein or derivatives thereof, but also in gene therapy, by using a gene expression vector including and expressing the human Herpesvirus 6 U94 gene.

Therefore, a third aspect of the present invention is a gene expression vector including and expressing the human Herpesvirus 6 U94 gene, for the aforementioned therapeutic uses of the invention.

According to a preferred embodiment, the gene expression vector is a herpes amplicon, however any gene expression vector suitable for use in gene therapy, particularly in humans, can be used in the implementation of the present invention.

It should be specified that both the human Herpesvirus 6 U94 protein and its encoding gene are known per se and that their sequences belong to the state of the art. In this regard, there can be mentioned, by way of example, the UniProtKB database (entries Q9WSZ6 and Q00683), and the papers by Gompels U A. et al., Virology, 1995 May 10; 209(1):29-51 and Mirandola P. et al., J Virol. 1998; 72:3837-3844.

The functional and/or therapeutic activity of the doses of active ingredients (proteins or nucleic acids) can be evaluated in vitro by various methods that measure the induction of HLA-G. For example, the immuno-modulating function of the HLA-G molecule expressed upon treatment with U94 or derivatives thereof or with the gene expression vector expressing the U94 gene can be evaluated in vitro through different systems that include, but are not limited to, the inhibition of the activation of NK cell lines against cell targets lacking HLA class I expression, such as for example the K562 cell line. The therapeutic dosage for the modulation of the expression of the HLA-G molecule can be extrapolated in vitro by using the U94 molecule or a derivative thereof or the gene expression vector expressing the U94 gene on cell lines representing the cell target of the action. The dosage also depends on the mode of administration. For example, in some applications, such as the treatment of bone marrow transplants, the cells in culture are treated with soluble suspensions of the active ingredients. The dosage to be used refers to the correct amount of the U94 molecule or a derivative or vector thereof that is able to cause enhancement/induction of expression of the HLA-G molecule in sufficient quantity to perform its immuno-inhibitory function. The therapeutic effect may include, but is, not limited to, the inhibition of the cytotoxicity of NK cells and T lymphocytes, the allogeneic T lymphocyte response, dendritic cell maturation, and the induction of regulatory T cells and IL-10-expressing dendritic cells. The precise dosage will depend on the number of cells to be treated, the size of the tissue and the nature of the same. The person skilled in the art is able to determine the correct dosage by routine experimentation.

Diseases and disorders or conditions associated with the need to induce an immune-tolerant state that can be treated according to the present invention include, but are not limited to, bone marrow transplantation, stem cell transplantation, transplantation of solid organs (such as for example kidney, liver, heart, lung, pancreas, intestine); autoimmune diseases (such as for example rheumatoid arthritis; multiple sclerosis), tumours, infertility.

The invention falls within the scope of immunosuppressive therapies to be implemented in the clinical setting. For this purpose, the U94 molecule or derivatives thereof or the gene expression vector expressing the U94 gene can be provided in the form of a kit or a pharmaceutical composition, with which a package leaflet can optionally be associated in the form regulated by law for the production, use and sale of pharmaceutical compounds, whose notes will include approval, use and sale for use in mammalian cells/tissues.

A further aspect of the present invention is an in vitro method for enhancing or inducing the expression of the HLA-G molecule in mammalian cell, tissue or organ cultures, which comprises administering to said cell, tissue or organ cultures the human Herpesvirus 6 U94 molecule or a derivative thereof, or a gene expression vector, preferably a herpes amplicon vector, expressing the human Herpesvirus 6 U94 gene, in an amount suitable to enhance or induce HLA-G expression in the treated cultures.

In one embodiment of the invention, the treated cell cultures are primary tumour-derived cells.

The experimental section that follows is provided for illustration purposes only and does not limit the scope of the invention as defined in the appended claims.

### Experimental Section

### U94 Production

M15 bacterial cells transformed with the pQE-rep plasmid were grown at 37° C. in LB containing 100 μg/ml ampicillin and 50 μg/ml kanamycin up to OD660 nm=0.4-0.6 and subsequently induced with 2 mM IPTG. After 1, 3 and 5 hours of induction at 37° C., the samples were collected by centrifugation and analysed for the presence of recombinant proteins. Cell pellets from 500 ml culture were suspended in 50 ml of lysis buffer (50 mM Tris-HCl pH 8, 2 mM EDTA, 1 mM DTT, 1 μg/ml aprotinin, 1 mM PMSF, 100 μg/ml lysozyme and 1% Triton X-100). After incubation for 30 minutes at room temperature, the suspension was cleared by sonication (three cycles of 15 s with a 15 s interval) and centrifuged for 15 minutes at 13,000 rpm at 4° C. Samples from supernatant (soluble fraction), pellet (insoluble fraction) and total lysate were analysed by SDS-PAGE. U94/REP protein was recovered mostly from the insoluble fraction, then the samples were solubilized in lysis buffer containing increasing molar concentrations of urea (2, 4, 6, 8 M). Each fraction was analysed by SDS-PAGE and Western blot. Complete solubilization of the protein was achieved with lysis buffer supplemented with 6 M urea. The fraction containing the recombinant protein was dialyzed to remove Triton X-100 residues and purified under denaturing conditions by hydroxyapatite chromatography, using the lysis buffer in a phosphate gradient. The sample was applied to the column after equilibration with lysis buffer without Triton, pH 6.8. The resin was washed with a 10-200 mM sodium phosphate buffer, and the protein was eluted with 500 mM phosphate. Each fraction was examined by spectroscopy at 280 nm and subsequently analysed by SDS-PAGE and Western blot. Endotoxin levels were tested and were below 0.5 EU/μg protein in all assays performed.

### HLA-G Induction

HUVEC cells were treated with 3 μg/ml recombinant U94/REP protein. Induction of HLA-G expression was determined by flow cytometry. Staining was performed with anti-HLA-G moAb (87 G moAb) (Exbio, Praha, Czech Republic), and isotypic controls (Exbio, Praha, Czech Republic). Analysis was carried out with a FACSCanto II cytometer and the FlowJo software. Results were expressed as MFI (mean fluorescence intensity).

### In Vitro Studies

### Cytotoxicity (MTT Assay)

100 μl of U937 cells at a density of 1×10⁶/m1 were plated and treated with U94 (3 μg/ml) for 24 hours. Subsequently, 10 μl of MTT (Sigma-Aldrich, Milan, Italy) were added for 4 hours at 37° C. The cells were lysed by adding 100 μl of MTT solvent. The plates were read at 570 nm using the plate reader (Victor, PerkinElmer, Waltham, USA).

### Immuno-Suppressive Activity of Treatment-Induced Molecules

Supernatants of HUVEC cell cultures treated or not treated with U94 were added to NK cell cultures (cell line NK92, ATCC-CRL2407), in a 1:5 ratio with the K562 target cells (ATCC-CCL243), characterized by the absence of HLA class I expression. NK cell activation status was determined by flow cytometry, by analysing CD107a expression. Briefly, the CD107a degranulation test was performed by incubating the cells for 3 hours at 37° C. with the Golgi Stop solution (Becton Dickinson) and subsequently by labelling with the anti-CD107a antibody (Becton Dickinson).

### HLA-G Induction by U94

Human MDA-MB 231 mammary carcinoma cells in which U94 expression was induced by an HSV-1-based amplicon were analysed by ELISA test for expression of soluble HLA-G secreted in the culture supernatants and by immunohistochemistry for expression of membrane-bound HLA-G.

The differences between the groups were assessed by the Mann Whitney U test. A p value <0.05 was regarded as a statistically significant value.

### Results

### In Vitro Studies

### U94-Induced HLA-G Expression

Cytotoxicity assessment has shown that the dose of U94 to be used is 3 μg/ml, as it induces the highest production of HLA-G with the lowest cellular toxicity (Table 1).

TABLE 1
Cytotoxicity analysis of the U94 protein

| U94 | HLA-G; % cells | Cytotoxicity; % |
|---|---|---|
| 3 μg/ml | 34.0 ± 2.5 | 5.2 ± 1.2 |
| 13 μg/ml | 25.2 ± 3.6 | 29.0 ± 4.6 |
| 20 μg/ml | 23. ± 4.1 | 49.0 ± 3.2 |

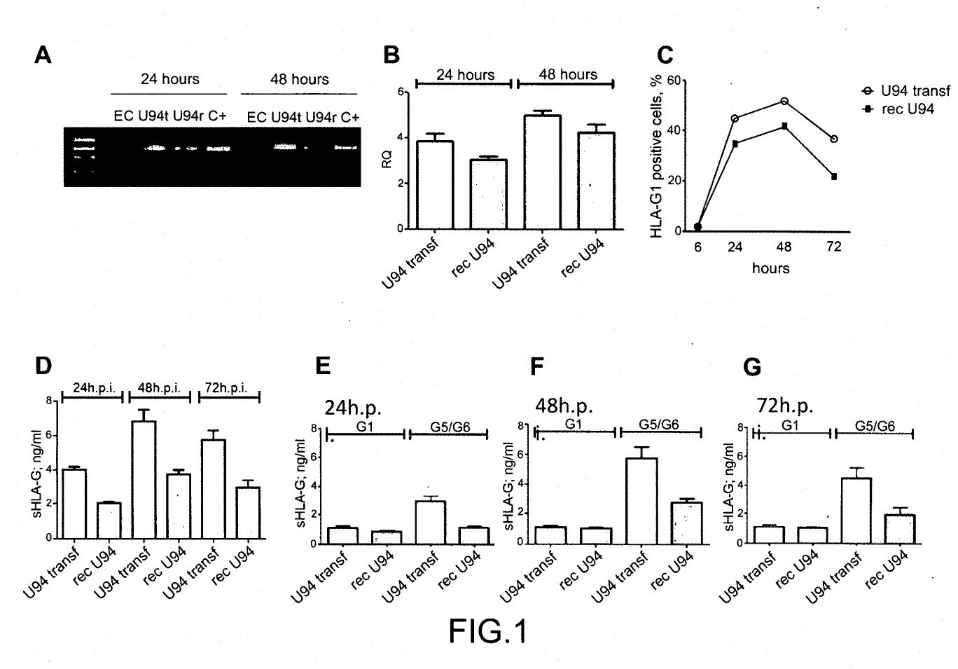

In the experiment shown in FIG. 1, HUVEC cells were treated with U94 (3 μg/ml). HLA-G expression has been shown as A, B) mRNA and C) membrane protein and D, E, F, G)) soluble. Following exposure to U94, HUVEC cells show increased expression of both the membrane-bound and the soluble form of HLA-G. In particular, after 24-48 hours from U94 transfection or addition of recombinant U94 to the culture medium of HUVEC cells there is an increase in the soluble form of HLA-G (HLA-G5/6) (FIG. 1 D, E, F, G).

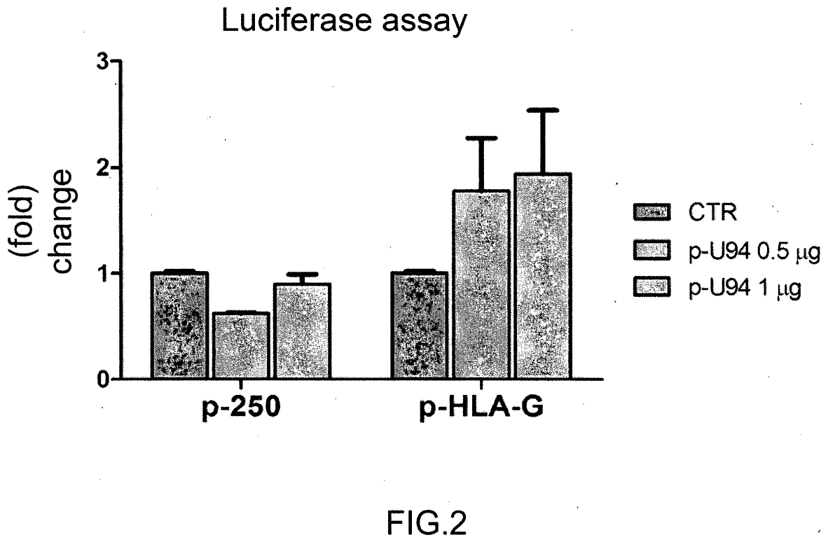

U94 activates the HLA-G gene promoter. In the pGL3-Basic reporter plasmid (Promega, Madison, Wis.) a PCR-generated fragment containing the HLA-G promoter sequence followed by the luciferase gene was cloned. U94 activates the HLA-G promoter. FIG. 2 shows the results of this experiment, in which HUVEC cells (10\hat{ }6 cells) were co-transfected with 0.5 μg p94 plasmid encoding the U94 viral gene or with the corresponding empty vector (CTR), together with 0.5 μg of pGL3-HLA-G1500 (pHLA-G) or pGL3-B250 (p250) and 0.2 μg of pRL-Renilla luciferase control reporter vector. Luciferase expression was assessed after 48 hours. The results are expressed as mean fold activation values±SD in duplicate samples from two independent experiments.

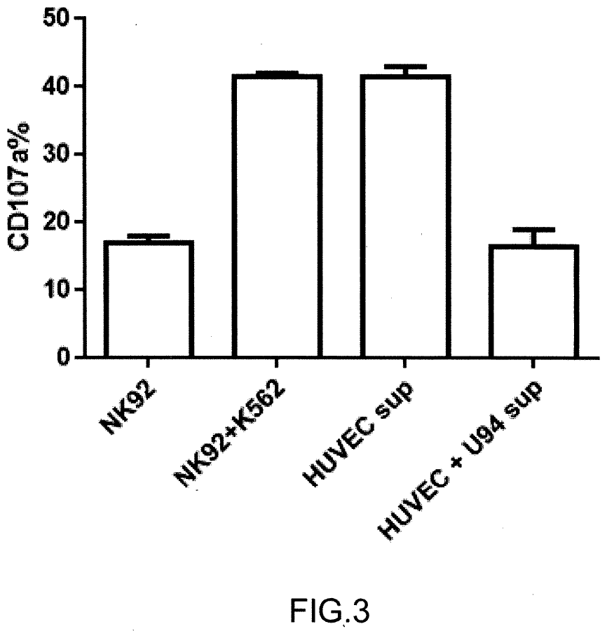

Supernatants of HUVECs treated or not treated with U94 were added to NK cell cultures (NK92 cell line), in the presence of K562 target cells. Activation status was analysed by flow cytometry (CD107a). Supernatants of U94-treated HUVEC cells containing HLA-G in its soluble form reduce the activation status of NK cells (FIG. 3). In contrast, supernatants of untreated HUVEC cells do not interfere with NK cell activation status. These results demonstrate the tolerogenic effect of U94-induced HLA-G molecules.

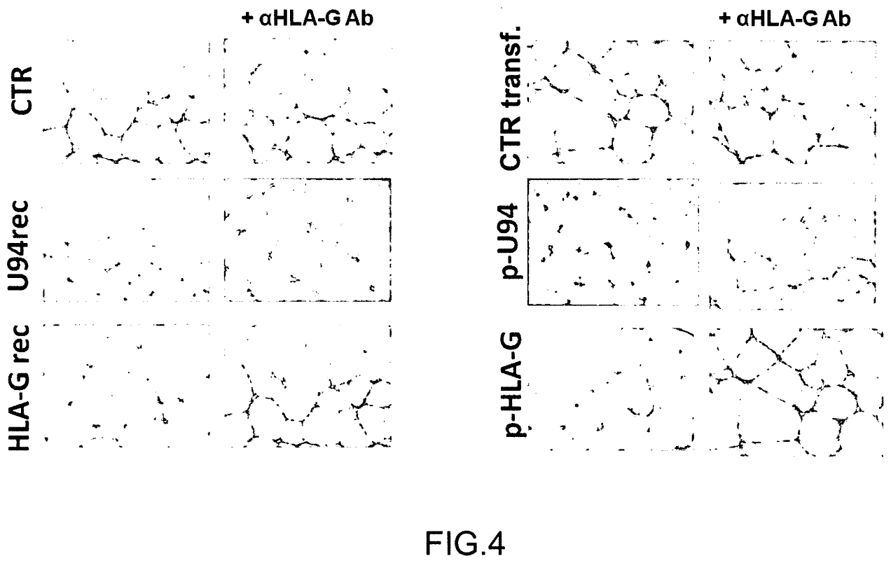

The ability of HUVEC cells to form capillaries is increased by transfection of the same cells with U94 or HLA-G, or by the addition of recombinant U94 or HLA-G proteins to the cell supernatants (FIG. 4). The addition of a specific antibody against HLA-G blocks the formation of capillaries in HUVEC cultures transfected with U94 or HLA-G or added with recombinant U94 or HLA-G (FIG. 4). This result confirms that induction of angiogenesis by U94 is mediated by the HLA-G molecule. In the experiment in FIG. 4, HUVEC cells were transfected with plasmids coding for HHV-6 U94 gene (pU94) or human HLA-G (pHLA-G) or treated with the recombinant U94 or HLA-G proteins. All samples were also tested with a specific antibody against HLA-G (αHLA-G Ab, 7.5 ng/ml). Images were obtained after a 24-hour incubation.

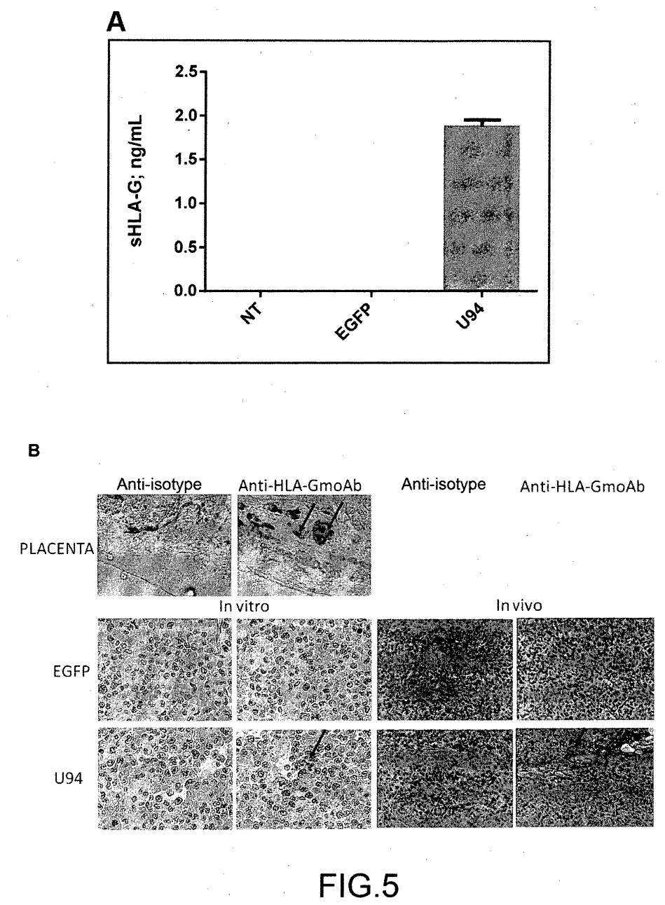

U94 expression in MDA-MB 231 cells induces HLA-G secretion in the supernatant (FIG. 5A). Membrane-bound HLA-G expression is evident in U94-expressing MDA-MB 231 cells both in a three-dimensional in vitro culture model (FIG. 5B) and in vivo in U94-expressing MDA-MB 231 cells inoculated in mouse (FIG. 5B). In particular, FIG. 5A shows the results of the analysis of soluble HLA-G levels in the culture supernatants of MDA-MB 231 cells in which U94 expression was induced by an HSV-1 based amplicon. NT: MDA-MB 231; EGFP: MDA-MB 231 with empty amplicon; U94: MDA-MB 231; EGFP: MDA-MB 231 with U94 amplicon. FIG. 5B shows the expression of membrane-bound HLA-G, as analysed by immunohistochemistry in U94-expressing MDA-MB 231 cells cultured in vitro in a three-dimensional system (left panel) or inoculated in mouse (right panel). Placental HLA-G expression was used as a positive control.

What is claimed is:

1-10. (canceled)

11. A method of administering an immuno-suppression and/or anti-inflammatory treatment to a subject in need thereof, the method comprising administering to the subject an amount of human Herpesvirus 6 U94 molecule or derivative thereof, the amount being effective in enhancing or inducing HLA-G expression in cells and/or tissues of the subject.

12. The method of claim 11, wherein the subject is a human being.

13. The method of claim 12, wherein the subject suffers from an autoimmune disease, a tumour disease, or infertility.

14. The method of claim 11, wherein the human Herpesvirus 6 U94 molecule or derivative thereof is administered as an adjuvant in a therapeutic immunosuppressive treatment in a bone marrow transplant, a stem cell transplant, or a solid organ transplant.

15. The method of claim 14, wherein the solid organ transplant is a kidney, liver, heart, lung, pancreas, or intestine transplant.

16. The method of claim 11, wherein the human Herpesvirus 6 U94 molecule or derivative thereof is co-administered with an immunosuppressive drug.

17. A method of administering an immuno-suppression and/or anti-inflammatory treatment to a subject in need thereof, the method comprising administering to the subject an amount of a gene expression vector including and expressing the human Herpesvirus 6 U94 gene, the amount being effective in enhancing or inducing HLA-G expression in cells and/or tissues of the subject.

18. An in vitro method for modulating the expression of the HLA-G molecule in a mammalian cell, tissue, or organ culture, the in vitro method comprising administering to said culture the human Herpesvirus 6 U94 molecule or a derivative thereof.

19. The in vitro method of claim 18, comprising administering to the mammalian cell, tissue, or organ culture an amount of the human Herpesvirus 6 U94 molecule or derivative thereof which is effective in enhancing or inducing HLA-G expression.

20. An in vitro method for modulating the expression of the HLA-G molecule in a mammalian cell, tissue, or organ culture, the in vitro method comprising administering to said culture a gene expression vector including and expressing the human Herpesvirus 6 U94 gene.

21. The in vitro method of claim 20, wherein the gene expression vector is a herpes amplicon vector.
